(19) United States
(12) Patent Application Publication
(10) Pub. No.: US 2022/0362123 A1
(43) Pub. Date: Nov. 17, 2022

(54) Disodium Lauryl Sulfosuccinate Composition

(71) Applicant: Colonial Chemical, Inc., South Pittsburg, TN (US)

(72) Inventors: Eugene D'AVERSA, South Pittsburg, TN (US); Robert N. COMBER, South Pittsburg, TN (US); Dennis ABBEDUTO, South Pittsburg, TN (US); Molly MCENERY, South Pittsburg, TN (US)

(21) Appl. No.: 17/630,415

(22) PCT Filed: Jul. 27, 2020
(86) PCT No.: PCT/US2020/043780

§ 371 Date: Jan. 26, 2022

Related U.S. Application Data
(60) Provisional application No. 62/878,906, filed on Jul. 26, 2019.

(51) Int. Cl. A61K 8/46; A61Q 5/02; A61K 8/60; A61K 8/44; C11D 1/83

(52) U.S. Cl. CPC A61K 8/466; A61Q 5/02; A61K 8/604; A61K 8/602; A61K 8/447; C11D 1/83; C11D 1/123

(57) ABSTRACT

A surfactant formulation comprised of: a sulfosuccinate in the range of 7 to 30 percent (active); an alkylpolyglucoside in the range of 5 to 30 percent (active); water in the range of 20 to 80 percent (active); and an optional preservative.

### BACKGROUND OF THE INVENTION

Sulfosuccinate surfactants can be used as both primary and secondary surfactants in a host of personal care applications including shampoos, body washes, facial washes, bubble baths and hand soaps. Disodium Lauryl Sulfosuccinate is one particularly common and popular sulfosuccinate, thanks to its natural derivation and providing superior foaming and thickening in surfactant based rinse off products. However, despite its popularity a major drawback of this surfactant is its physical form. Even at modest surfactant activities between 30 and 45 percent, disodium lauryl sulfosuccinate is a white, opaque thick paste. Difficulty working with disodium lauryl sulfosuccinate is compounded by the fact that it is sensitive to high temperatures and cannot be stored for extended periods at high temperature or incorporated into very high temperature processes, which would normally allow for quick incorporation of the material into formulations.

Some examples and manufacturers of Disodium Lauryl Sulfosuccinate that are difficult to work with in their present form include, but are not limited to: Mackanate LO (Solvay Novecare), Cycloryl DLS (Cedar Concepts), SCT-725 (Coast Southwest), Miconate DLS(N) (Miwon Corporation), Galaxy LSS P (Galaxy Surfactants), Pel-Surf L (Ele Corporation) and Cola®Mate LA-40 (Colonial Chemical, Inc.).

### SUMMARY OF THE INVENTION

An embodiment of the present invention is a surfactant formulation. An example is a surfactant formulation that comprises a sulfosuccinate in the range of 7 to 30% (active); an alkylpolyglucoside in the range of 5 to 30% (active); and water in the range of 20 to 80% (active).

The formulation may also comprise a preservative. When present, embodiments of the invention include a preservative present in an amount of up to 2 (active) %.

Another embodiment of the present invention is a surfactant formulation that comprises a sulfosuccinate in the range of 20 to 80 wt %; an alkylpolyglucoside in the range of 10 to 60 wt %; and water in addition in the range of 1 to 50 wt %.

In aspects of the invention, the formulation may comprises other materials, such as at least one of an antioxidant, a pH adjuster, and a rheology modifier.

In other aspects of the present invention, the formulation is pourable with a viscosity range of 10-1000 cps.

In other aspects of the invention, the sulfosuccinate is of the following formula:

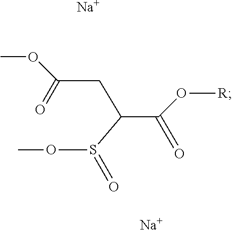

wherein R is C₆-C₁₄ alkyl.

In other aspects, the sulfosuccinate is of the following formula:

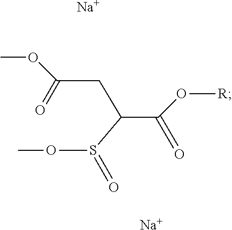

wherein R is C₁₂ alkyl; disodium lauryl sulfosuccinate.

In other aspects, the sulfosuccinate may be a mixture of more than one sulfosuccinate, and R is independently chosen from C₆ alkyl, C₈ alkyl, C₁₀ alkyl and C₁₂ alkyl.

In other aspects, the alkylpolyglucoside has an alkyl group at the glycosidic position that is a C₆, C₈, C₁₀, C₁₂ alkyl or a mixture of thereof.

Another embodiment of the present invention is a surfactant-based cleaner product made from a formulation of the present invention. An example of the product is a personal care product. Another example is a shampoo, body wash, or facial wash. Yet another example is a household, institutional and industrial cleaning product.

Additional advantages and embodiments of the present invention will be set forth in the description which follows, and in part will be obvious from the description, or can be learned by practice of the invention. The advantages and embodiments of the present invention will be realized and attained by means of the elements and combinations particularly pointed in the appended claims. It is to be understood that both the foregoing general description and the following more detailed description are exemplary and exemplary only and are not restrictive of the invention, as claimed.

### DESCRIPTION OF THE INVENTION

The present invention meets a long felt need by providing a disodium lauryl sulfosuccinate that is user friendly, easy to work with, and in some embodiments a form that can be dispensed with relative ease in manufacturing with a pump.

Thus, embodiments of the present invention provides superior disodium lauryl sulfosuccinate intermediate products. The products of the present invention make it easier to take advantage of the excellent properties of disodium lauryl sulfosuccinate, while providing a more user-friendly version that makes production of the final product easier. In embodiments of the present invention, the sulfosuccinate is a disodium lauryl sulfosuccinate. In some embodiments, the disodium lauryl sulfosuccinate is a blend using Cola®Mate LA-40 manufactured and sold by Colonial Chemical, Inc., South Pittsburgh, Tenn.

In some “big-batch” manufacturing processes (a shampoo formulation, for example), Cola®Mate LA-40 may have to be scooped out of a drum or other container into a heated tank of water and other surfactants or additives. Alternately, the product may be heated in the package, with some risk of degrading the product over time. Since the present invention renders the Cola®Mate LA-40 easier to work with, when in the inventive form it may be pumped into the batch. This invention potentially allows for “cold-mix” processing, removing the time-consuming and costly steps of heating and cooling the bulk formulation.

Embodiments of the present invention relates to the finding that sulfosuccinates can be blended with alkylpolyglucosides and water to give flowable, pumpable solutions and in some embodiments clear, pourable solutions that retain many of the attributes of the parent sulfosuccinates.

Thus, one embodiment of the present invention is a blend of a sulfosuccinate and an alkylpolyglucoside. This embodiment may further include a preservative and added water.

### Sulfosuccinates

The sulfosuccinates of the present invention can be present in a range of about 20-80 wt %, and any range or amount in-between. For example, they may also be present in a range of about 30-70 wt %. They may also be present in an amount of from about 40-60%. They may also be present in an amount of about 20, 25, 30, 35, 40, 45, 50, 55, 60, 65, 70, 75, or 80 wt %.

They may also be present as sulfosuccinate blends.

As stated above, example of the sulfosuccinate of the present invention is Cola®Mate LA-40. Cola®Mate LA-40 is a light colored, mild sulfosuccinate derived from natural lauryl alcohol. Cola®Mate LA-40 has more than 75% renewable carbon content due to the vegetable origin of the lauryl alcohol used in the product. Cola®Mate LA-40 is sulfate free, ethoxylate free and 1,4-dioxane free — making it ideal for natural based, sulfate-free formulations. Cola®Mate LA-40 provides dense, voluminous foam when used to formulate personal cleansers, is extremely mild to eyes and is useful in sensitive skin cleaners. It provides good viscosity response when formulated with other anionic surfactants. Typical applications of the formulations include shampoos, bubble baths, body washes and gels, face washes, hand soaps, etc. However, it exhibits a very high Krafft Point of approximately 50° C. and in its typical room temperature form of a soft, white, immobile paste, it is hard to handle.

A sulfosuccinate of the present invention is shown below:

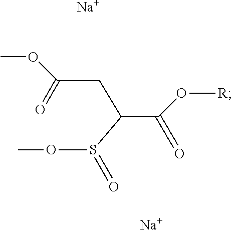

wherein R is C₆-C₁₄ alkyl.

Another embodiment of a sulfosuccinate of the present invention is shown below:

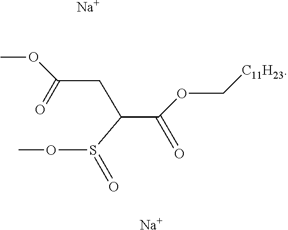

An example of a successful use of a sulfosuccinate blend of the present invention is its part in a blend of surfactants for use as a base for high-foam personal care products. An example is an eco-friendly blend that is free of alcohol alkoxylates, diethanolamides, lauryl sulfates, parabens, and/or formaldehyde.

Examples of the present invention also include the following sulfosuccinate blends:

| Material | Wt % (active) |
|---|---|
| Di sodium Lauryl Sulfosuccinate | 7-30 |
| C₈-C₁₀ alkyl glucoside | 5-30 |
| Water | 20-80 |
| Preservative | 0.01-2.0 |

### Alkylpolyglucosides

The alkylpolyglucosides of the present invention can be present in a range of about 10-60 wt %, and any range or amount in-between. For example, they may also be present in a range of about 20-50 wt %. They may also be present in an amount of from about 25-35%. They may also be present in an amount of about 10, 15, 20, 25, 30, 35, 40, 45, 50, 55, or 60 wt %.

They may also be present as alkylpolyglucoside blends.

An alkylpolyglucoside such as, for example, capryl glucoside, decyl glucoside, coco-glucoside, or lauryl glucoside is used in the composition of the present invention. Alkylpolyglucosides are commercially available from a number of sources and include, but are not limited to: Glucopon™ surfactants and Plantaren surfactants, both from BASF SE (Ludwigshafen, Germany), Milcosides Surfactants from Galaxy Surfactants, Ltd. (Maharashtra, India) EcoSense Surfactants from The Dow Chemical Company (Midland, Mich.), and Oramix Surfactants from Seppic, Inc. (Paris, France).

Examples of an alkylpolyglucosides used herein, refers to a compound having the following structure:

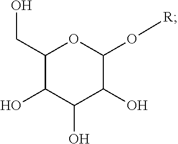

wherein R is an C₂-C₂₀ alkyl, more preferably a C₄-C₁₄ alkyl. In certain embodiments, R may be a Cₙ alkyl, wherein n is 4, 5, 6, 7, 8, 9, 10, 11, 12, 13, 14, 15, 16, 17, 18, 19, or 20. In certain embodiments, R is an unsubstituted alkyl; it is nonetheless anticipated that R may be a substituted alkyl in various embodiments. An example of the C₈-C₁₀ alkyl glucoside is Glucopon 215 available from BASF. As sold, many of the alkyl polyglucosides contain a significant amount of the disaccharide polymer depicted below:

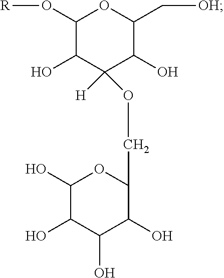

wherein R is described above.

### Preservatives

To adequately preserve these products and prevent microbial growth, a preservative can optionally be included in the compositions disclosed herein. One of ordinary skill in the art can readily select suitable preservative, including those available in the haircare and cosmetic industry.

Acceptable preservatives include, without limitation, benzoic acid, benzyl alcohol, butylparaben, propylparaben, methyparaben, DMDM Hydantoin, potassium benzoate, methylisothiazolinone, methylchloroisothiazolinone, phenoxyethanol, ethylhexylglycerin, quaterium-8, quaterium-14, quaterium-15, triclosan, and zinc salicylate.

An example of a preservative of the present invention is Kathon CG, available from Dow. The active ingredients of KATHON CG preservative are two isothiazolinones, identified by the IUPAC system of nomenclature as: 5-chloro-2-methyl-4-isothiazolin-3-one and 2-methyl-4-isothiazolin-3-one.

### Thickening Agent

Those skilled in the art of personal care formulating realize that formulations can be optionally thickened with relatively low amounts of suitable thickeners or rheology modifiers, including those that allow for cold process.

The thickeners/rheology modifiers can be used to adjust the rheological characteristics of the composition for better feel, in-use properties and the suspending stability of the composition. For example, the rheological properties are adjusted so that the composition remains uniform during its storage and transportation. Any suitable rheology modifier can be used.

The one or more rheology modifier may be selected from the group consisting of polyacrylamide thickeners, cationically modified polysaccharidea, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobic ally modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and) polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquatemium-37 (and) hydrogenated polydecene (and) trideceth-6; polyurethane-39; sodium acrylate/acryloyldimethyltaurate/dimethyl acrylamide; crosspolymer (and) isohexadecane (and) polysorbate 60; sodium polyacrylate.

Another example of a thickener that may be used in connection with the present invention is a polyether ester thickening agent disclosed in U.S. Pat. No. 5,192,462, assigned to Croda, Inc.

### Additives

One of ordinary skill in the art would recognize that in the instance of preparing products with the surfactant compositions of the present invention, further additives can be used. These may include, but are not limited to, antioxidants, fatty substances/oils, softeners, emulsifiers, screening agents, fragrances, dyes, colorants, pigments or nanopigments, light stabilizers, insect repellents, antibacterial agents, or any other ingredients usually formulated into such products. The necessary amounts of the additives can, based on the desired product, easily be chosen by a skilled artisan in this field.

### Water

Water in addition may be present in the formulations, with the appropriate amount readily determined by one of ordinary skill in the art. Examples of amounts may range from about 1 to 50 wt % or more.

Examples of the formulation of the present invention are clear or slightly hazy, free-flowing or pumpable liquids and free of alcohol alkoxylates and free of ethylene oxide (EO) or dioxane. Additionally, examples of the formulations of the present invention retained key properties of the parent sulfosuccinate and alkylpolyglucosides such as overall viscosity and foaming when used as intermediates in further formulations.

Example formulations of the present invention are shown below in Table 1.

TABLE 1

| Examples of | | | |
|---|---|---|---|
| flowable, pumpable | | | |
| blends of the current | Blend 1 | Blend 2 | Blend 3 |
| invention | (Wt %) | (Wt %) | (Wt %) |
| Disodium Lauryl | 40 | 50 | 60 |
| Sulfosuccinate (37%) | | | |
| Glucopon 215, 50% | 25 | 30 | 35 |
| (Caprylyl, Decyl | | | |
| Glucoside) | | | |
| Water | 34.0-34.96 | 19.0-19.96 | 4.0-4.96 |
| Preservative | 0.04-1.0 | 0.04-1.0 | 0.04-1.0 |

An example of the utility of the current invention is shown by the formulations in Table 2. In these formulations, Blend 1 was used as a flowable, pourable blend containing Disodium Lauryl Sulfosuccinate (Cola®Mate LA-40 sold by Colonial Chemical, Inc.) Suga®Nate 160NC is Sodium Laurylglucosides Hydroxypropylsulfonate (sold by Colonial Chemical, Inc.). Cola®Teric CBS-HP is Cocamidopropyl Hydroxysultaine (sold by Colonial Chemical, Inc.).

TABLE 2
Cold process shampoo formulations using flowable, pourable blends of the current
invention (wt %)

| Ingredient | A | B | C | D |
|---|---|---|---|---|
| Water | 65.80 | 65.80 | 56.80 | 62.80 |
| Suga ®Nate | 18.00 | 18.00 | 18.00 | 12.00 |
| 160NC | | | | |
| Cola ®Mate LA- | 6.00 | — | — | — |
| 40 | | | | |
| Blend 1 | — | 6.00 | 15.00 | 15.00 |
| Cola ®Teric | 9.00 | 9.00 | 9.00 | 9.00 |
| CBS-HP | | | | |
| Euxyl K712 | 1.00 | 1.00 | 1.00 | 1.00 |
| Fragrance | 0.2 | 0.2 | 0.2 | 0.2 |
| Citric Acid 50% | qs to pH 5.0-5.5 | qs to pH 5.0-5.5 | qs to pH 5.0-5.5 | qs to pH 5.0-5.5 |
| Appearance | Clear liquid | Clear Liquid | Clear Liquid | Clear Liquid |
| Surfactant Solids | 14.0 | 13.5 | 16.4 | 14.0 |
| pH | 5.06 | 5.02 | 5.05 | 5.03 |
| Viscosity, cP | 6,709 | 157 | 137 | 62 |
| Ross Miles Foam | | | | |
| Height, mm (1%) | | | | |
| Immediate | 190 | | 190 | |
| 1 Minute | 165 | | 165 | |
| 5 Minutes | 160 | | 160 | |

The data is Table 2 show that Disodium Lauryl Sulfosuccinate, normally a thick, unpumpable white paste (used in that product form in making Formula A, which required heating) can be easily incorporated into cold process formulas as a thin, pourable clear or slightly hazy liquid (as delivered in Blend 1 in formulas B, C and D in Table 2). Comparing Formulas A and C, it can be seen that using a thin, pumpable blend to deliver disodium lauryl sulfosuccinate results in a significant viscosity drop of the formulation. Formula A, made with heating and using the paste form of disodium lauryl sulfosuccinate had a viscosity of 6709 cP. Formula C, made without heating, with a cold process and using a thin, pourable version of disodium lauryl sulfosuccinate, had a viscosity of only 137 cP. Both formulations performed well in Ross Miles foam studies and had identical initial and final foam heights, demonstrating the very desirable foam building characteristics of disodium lauryl sulfosuccinate.

Those skilled in the art of personal care formulating realize that formulations can be thickened with relatively low amounts of suitable thickeners or rheology modifiers, including those that allow for cold processing. The formulas shown in Table 3 demonstrate that formulations made with pourable, pumpable blends of disodium lauryl sulfosuccinate can be readily thickened using common thickening agents or rheology modifiers used in personal care. The thickening agent used in Table 3 is Versathix (a mix of PEG-150 Pentaerythrityl Tetrastearate, PPG-2 Hydroxyethyl Cocamide and water, sold by Croda, Inc.). The latter, when added cold to either of the formulas in Table 3, produces stable shampoos with viscosities 8000-8500 cP. Note that Shampoo E is essentially the same as Shampoo F because the components of Blend 1, Glucopon 215 and Cola Mate LA-40, are added in the same ratios to Shampoo E that they occur in Blend 1 in Shampoo F. As formulated, both had identical viscosities and foam height performance, but Shampoo A required warming.

TABLE 3
Adjusting viscosity of cold process shampoos blends made with
pourable, pumpable disodium lauryl sulfosuccinate (wt %)

| | | |
|---|---|---|
| Water | 67.15 | 58.75 |
| Glucopon 215 UP | 6.00 | |
| Cola ®Mate LA-40 | 9.60 | |
| Blend 1 | | 24.00 |
| Cola ®Teric CBS-HP | 14.00 | 14.00 |
| Cola ®Liquid DC | 2.00 | 2.00 |
| Versathix-LQ-(MH) | 1.25 | 1.25 |
| Citric Acid 50% | qs to pH 6.0 | qs to pH 6.0 |
| Appearance | Clear | Clear |
| Surfactant Solids, % | 16.5 | 16.5 |
| pH | 5.94 | 5.92 |
| Viscosity, cP | 8,210 | 8,370 |
| Ross Miles Foam Height, mm | | |
| (1%) | | |
| Immediate | 180 | 180 |
| 1 Minute | 160 | 160 |
| 5 Minute | 155 | 155 |

As noted above, a number of thickeners or rheology modifiers are available for adjusting the final viscosity of personal care shampoos, body washes and facial washes. Some allow cold processing and some need to be heated to be incorporated into the formula. Not all will allow the claim of EO-free or dioxane free. Notwithstanding, blends of the current invention allow for incorporating the very desirable high foaming sulfosuccinate surfactants, specifically disodium laury sulfosuccinate, in a liquid, pumpable form, allowing for the possibility of highly natural, cold process personal care cleansing products that are EO-free, dioxane free, sulfate free with significantly decreased manufacturing times.

As used herein, the singular forms “a,” “an” and “the” include plural referents unless the context clearly dictates otherwise. Thus, for example, reference to “a surfactant” includes mixtures of two or more such surfactants.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein.

All publications mentioned herein are incorporated herein by reference in their entirety to disclose and describe the methods and/or materials in connection with which the publications are cited.

1. A surfactant formulation comprising:
a. a sulfosuccinate in the range of 7 to 30% (active);
b. an alkylpolyglucoside in the range of 5 to 30% (active); and
c. water in the range of 20 to 80% (active).

2. The formulation of claim 1, further comprising a preservative, the preservative being present in an amount of up to 2 (active) %.

3. A surfactant formulation comprising:
a. a sulfosuccinate in the range of 20 to 80 wt %;
b. an alkylpolyglucoside in the range of 10 to 60 wt %;
c. water in addition in the range of 1 to 50 wt %.

4. The formulation of claim 3, further comprising a preservative.

5. The formulation of claim 1, further comprising at least one of an antioxidant, a pH adjuster, and a rheology modifier.

6. The formulation of claim 1, wherein the formulation is pourable with a viscosity range of 10-1000 cps.

7. The formulation of claim 1, wherein the sulfosuccinate is of the following formula: 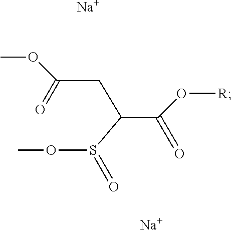
wherein R is C₆-C₁₄ alkyl.

8. The formulation of claim 1, wherein the sulfosuccinate is of the following formula: 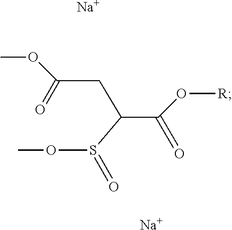
wherein R is C₁₂ alkyl; disodium lauryl sulfosuccinate.

9. The formulation of claim 7 wherein the sulfosuccinate is a mixture of more than one sulfosuccinate, and R is independently chosen from C₆ alkyl, C₈ alkyl, C₁₀ alkyl and C₁₂ alkyl.

10. The formulation of claim 1 where the alkylpolyglucoside has an alkyl group at the glycosidic position that is a C₆, C₈, C₁₀, C₁₂ alkyl or a mixture of thereof.

11. A surfactant-based cleaner product, comprising a formulation of claim 1.

12. The product of claim 11, formulated into a personal care product.

13. The product of claim 12, formulated as a shampoo, body wash, or facial wash.

14. The product of claim 11, formulated as or into a household, institutional and industrial cleaning product.

15. The formulation of claim 1, wherein the formulation is ethylene oxide and dioxane-free.
